(19) United States
(12) Patent Application Publication
(10) Pub. No.: US 2006/0165661 A1
(43) Pub. Date: Jul. 27, 2006

(54) Pediocin-producing pediococci

(75) Inventors: Gea Speelmans, Wageningen (NL); Adrianus Johannes Maria Vriesema, Houten (NL); Simone Dinanda Elbertje Oolhorst, Wageningen (NL)

Correspondence Address: THE WEBB LAW FIRM, P.C., 700 KOPPERS BUILDING, 436 SEVENTH AVENUE, PITTSBURGH, PA, 15219, US

(73) Assignee: N>V> Nutricia

(21) Appl. No.: 10/560,864

(22) PCT Filed: Jun. 14, 2004
(86) PCT No.: PCT/NL04/00420

§ 371 Date: Mar. 28, 2006

(30) Foreign Application Priority Data
Jun. 13, 2003 (EP) 03076324.4

(51) Int. Cl. A61K 35/74

(52) U.S. Cl. 424/93.4; 424/93.45

(57) ABSTRACT

There are provided pediocin-producing pediococci that have an enhanced survival property in the small intestine. The pediococci comprise in particular Pediococcus acidilactici isolated from human faeces. The pediococci are suitable for use in the gastrointestinal tract of humans to provide a health-promoting action, in particular against infection by multi-resistant pathogens. Also provided herein are compositions incorporating such pediocin-producing pediococci.

### TECHNICAL FIELD OF THE INVENTION

The present invention relates to pediocin-producing pediococci, in particular Pediococcus acidilactici isolated from faeces, for use in the gastrointestinal tract of humans to provide a health-promoting action, in particular against infections by multi-resistant pathogens.

### BACKGROUND OF THE INVENTION

Intestinal diseases such as intestinal infections are usually caused by bacteria or viruses. Bacterial infections can be treated with synthesised antibiotics. Synthesised antibiotics are chemical compounds that will kill pathogenic bacteria. However, a problem encountered with such antibiotics is that after regular administration to the patient, the organism begins to build up a certain resistance to said antibiotics, thus requiring either the use of a stronger synthesised antibiotic or the use of an alternative solution to treat the infection.

Alternative solutions can be presented by the use of probiotics. Probiotics are viable bacteria that beneficially affect the host by improving its intestinal microbial balance. These bacteria can have a prophylactic and/or a therapeutic effect on intestinal diseases, such as intestinal infections. Thus, when these micro-organisms are administered to humans or animals, they can compete with pathogenic bacteria for nutrients and/or inhibit adhesion sites on the intestinal wall, as a result of which the number of pathogenic bacteria will decrease and infections are prevented or reduced. Further, probiotics produce organic acids, thereby lowering the pH, and thereby resulting in an antipathogenic action.

Some bacteria can produce anti-microbial proteins or peptides, called bacteriocins. These anti-microbial components can inhibit the growth of bacteria by impairing the cytoplasmic membrane of sensitive bacteria resulting in disturbances of the intra-cellular homeostasis.

Yet, not all bacteriocins have the same spectrum of activity. For example nisin, which is a cationic peptide antibiotic produced by Lactococcus lactis, has a very broad spectrum of activity, that is one which is detrimental to both beneficial strains such as lactobacilli and bifidobacteria as well as pathogenic strains in the intestine.

Accordingly, there is a need for a material has a selective activity to the pathogenic species rather than to the beneficial species present in the intestine. By pathogenic strains, it is meant Gram-positive bacteria, in particular those of the enterococci-type like Enterococcus faecalis, Enterococcus faecium, but also Listeria monocytogenes and mixtures thereof as well as Gram-negative bacteria, in particular those belonging to the genera Klebsiella, Pseudomonas and Shigella.

Further, with the emergence and wide geographic spread of multidrug-resistant Gram-positive pathogens, the necessity for alternative antibacterial agents has become urgent. Typical of such multidrug-resistant Gram-positive pathogens are the multi-resistant pathogens, in particular the vancomycin-resistant enterococci, hereinafter called VRE. The problem of infection caused by such VRE is even more acute in hospitals, wherein patients weakened by surgery or diseases are more sensitive to such pathogens. Accordingly, there is also a need for a material that is effective against VRE.

Recently, the action of nisin has been studied in “Journal of Antimicrobial Chemo-therapy” (1998) 41, 341-347 and was found to have a wide and powerful bactericidal effect, in particular against vancomycin-resistant isolates of E. faecium and E. faecalis. However, for the reasons stated above, nisin is not a desired material for use herein due to its non-selective activity spectrum, thus being to some extent detrimental to the intestinal floral equilibrium.

Besides VRE, the presence of non-vancomycin-resistant enterococci in the gastrointestinal (GI) tract can also be a cause of intestinal diseases. Accordingly, there is a need for a material that is also effective against non-vancomycin-resistant Enterococcus, especially E. faecalis as well as E. faecium.

Accordingly, there is also a need for a material which is effective against the pathogenic species present in the intestine, in particular against the pathogenic Gram-positive species, more in particular against enterococci, including VRE, Enterococcus faecalis, Enterococcus faecium; against Listeria monocytogenes and mixtures thereof as well as against the pathogenic Gram-negative species, in particular those belonging to the genera Klebsiella, Pseudomonas, Shigella and mixtures thereof, whilst not being detrimental to the intestinal flora.

Still another problem arising with the presence of pathogens is the overgrowth of said pathogen in the gut, which causes diarrhea as well as secondary associated disorders. Typical of such secondary disorder associated with overgrowth of pathogens are water disturbances, mineral balance disturbances, malnutrition, dysfunctioning of tissues, dysfunctioning of organs and dysfunctioning of organism. The disturbances and malnutrition can in turn cause one or more of the following: nausea, vomiting, sickness, but also endotoxemia, as well as sepsis (“blood-poisoning”), whereas the dysfunctioning mentioned above can in turn cause one or more of the following: infections, ulcers, bad wound healing, both in the gut as well as topically, each of which can be fatal.

Accordingly, there is also a need for a material that is effective against overgrowth of pathogens and thereby the associated secondary disorders.

Pediocin-producing Pediococcus strains are known in the art. They are described in FEMS Microbiology Reviews 24 (2000) 85-106 by S. Ennahar et al., as well as in Natural Food Antimicrobial Systems 19 (2000) 525-566 by B. Ray and K. W. Miller. However, both publications describe pediocin-producing pediococci as being isolated from meat or plant. In that respect, it has been found that such isolates do not optimally perform when used in the human GI tract. Hence, the overall survival in the GI tact has been found lower than with isolates of the invention.

Erkkilä et al., Meat Science 55 (2000) 297-300, have screened meat starter cultures and concluded that Lactobacillus curvatus strain RM10 and Pediococcus acidilactici strain P2 from commercial meat starter cultures can survive acid and 0.3% bile salts to an extent of 10% and would therefore be candidates for use as probiotics. However, these survival rates are insufficient for use in an effective probiotic composition, especially for effectively controlling pathogenic micro-organisms in the GI tract.

### DESCRIPTION OF THE INVENTION

It has now surprisingly been found that strains of Pediococcus that have a specific survival rate in the gastrointestinal tract, preferably strains of pediocin-producing Pediococcus, in particular strains of P. acidilactici, more in particular the P. acidilactici strains isolated from human faeces, fulfil the above-mentioned needs. P. acidilactici isolated from human faeces has been deposited at the Belgian Co-ordinated Collections of Microorganisms, Laboratorium voor Microbiologie Gent (BCCM™/LMG) on 24 Apr. 2003 under No. LMG P-21927.

Pediococcus strains of the invention and its pediocin have been found to be particularly effective for inhibiting, in the gastrointestinal tract, the growth of pathogenic Gram-positive strains, in particular of the enterococci type, preferably selected from vancomycin-resistant enterococci, Enterococcus faecalis, Enterococcus faecium, and Listeria monocytogenes. Furthermore, the Pediococcus strains of the invention, in particular pediocin-producing Pediococcus strains, have been found to be particularly effective in inhibiting in the gastrointestinal tract the adhesion of pathogenic Gram-negative strains, in particular those selected from the genera Klebsiella, Pseudomonas and Shigella.

It is an aspect of the present invention to provide the use of pediococci for the manufacture of a composition for inhibiting pathogens strains in the gastrointestinal tract, wherein the pediococci are characterised by a survival rate as per the Survival Rate Test defined herein of at least 80%, and/or are isolated from human faeces

It is another aspect of the present invention to provide the use of such pediococci and/or bacteriocin, preferably pediocin, produced from pediococci for the manufacture of a composition for inhibiting the growth of Gram-positive strains, especially of the enterococci type, in the gastrointestinal tract

It is a further object of the present invention to provide the use of such pediococci for the manufacture of a composition for inhibiting in the gastrointestinal tract the growth and/or adhesion of Gram-negative strains.

In another aspect of the present invention, there is provided an isolated pediocin-producing Pediococcus strain exhibiting excellent survival in the gastrointestinal tract, as well as a probiotic component which comprises such a strain.

In a further aspect of the present invention, there is provided a health-promoting action composition comprising said probiotic component, and preferably with a component selected from pediocin as herein defined, additional probiotics, prebiotics, immunoglobulins, and mixtures thereof.

The pediococci of the invention are characterised by a survival rate as per the Survival Rate Test defined below of at least 80%, preferably of at least 90%, most preferably of at least 96%. It is particularly preferred that the survival rate according to the Survival rate Test exceeds 100% (indicating that the organism is able to grow in the gastrointestinal tract).

Indeed, it has surprisingly been found that the pediococci according to the present invention present an overall survival in the GI-tract, in particular in the small intestine after the passage in the stomach, that is superior to pediococci isolated from plant or meat. This is demonstrated hereinafter in Table 3 of the examples.

Survival Rate Test

The Survival Rate Test is designed to evaluate strains which will survive both in the stomach and small intestine. For the purpose of the invention, the strains encompassed by the present invention are those which after contact for 3 h at 37° C. under anaerobic conditions in a medium that represents the stomach, and thereafter exposed for 3 h at 37° C. under anaerobic conditions to a medium that represents the small intestine have a survival rate of at least 80%, preferably of at least 90%, and more preferably of at least 96% in the small intestine.

The Survival Rate Test is performed as follows:

The bacteria to be tested are grown in MRS for 24 hours and subsequently re-inoculated for 18 hours in MRS at 37° C. 1 ml of the grown culture is added to 9 ml of reconstituted stomach medium, consisting of 8.3 g/l bacteriological peptone, 3.1 g/l NaCl, 0.11 μl CaCl₂, 1.1 g/l KCl, 0.6 g/l KH₂PO₄, 22.2 mg/l pepsin and 22.2 mg/l lipase, pH 3.0 (measured at 20° C.). The bacteria are incubated for 3 hours at 37° C. in the reconstituted stomach medium. Afterwards 1 ml of the incubated stomach medium with the bacterium is mixed with 9 ml of reconstituted small intestine medium and incubated for another 3 hours at 37° C. The reconstituted small intestine medium consists of 5.7 g/l bacteriological peptone, 1.25 μl NaCl, 0.055 g/l CaCl₂, 0.15 μl KCl, 0.68 μl KH₂PO₄, 1.0 g/l NaHCO₃, 0.3 g/l Na₂HPO₄, 0.7 g/l glucose, 20.3 g/l pancreatin and 5.5 μl bile, pH 6.5 (measured at 20° C.). Samples are taken at t=0, 3, and 6 hours and plated on MRS agar to determine the colony forming units.

To determine the survival rate in the small intestine alone, the survival rate in the stomach can be set at 100% as an approximation. The % survival in the small intestine is determined as 100×(cfu's present in 10 ml small intestine medium/cfu's present in 1 ml stomach medium).

Alternatively, pediocin produced from the pediococci strains of the present invention will be obtained by purification from the culture supernatant of the pediocin-producing pediococci whilst the pediocin-producing pediococci will be obtained from the culture itself or via downstream processes which are known in the art such as centrifugation, filtration, washing and/or drying steps, etc.

Preferred pediocin-producing pediococci are those of the type selected from Pediococcus acidilactici, Pediococcus penitosaceus, and mixtures thereof. Most preferred pediocin-producing pediococci are those of the Pediococcus acidilactici type.

Pediococcus acidilactici LMG P-21927

Pediococcus acidilactici LMG P-21927, that is isolated from human faeces, is a most preferred pediocin-producing Pediococcus strain for the purpose of the invention. Also, the antimicrobial compound produced by the strain, i.e. pediocin has been found beneficial for inhibiting in the gastrointestinal tract strains selected from Gram-positive strains.

By “isolated” is meant that the pediocin-producing pediococci are separated from their source, such as human faeces. This can be done according to the isolation method as described further below.

For the purpose of the present invention, when Pediococcus acidilactici LMG P-21927 is mentioned, this also encompasses its replicates, mutants and its derivatives.

By “replicate” is meant any biological material that represents a substantially unmodified copy of the materials, such as material produced by growth of microorganisms.

By “mutant” and “derivative” is meant material created from the biological material and which is substantially modified to have new properties, for example caused by heritable changes in the genetic material. These changes can either occur spontaneously or be the result of applied chemical and/or physical agents and/or by recombinant DNA techniques. However, it is preferred that such mutants and derivatives still produce pediocin.

Interaction between pathogenic bacteria and the host cells initiates infectious diseases. Attachment of these pathogens to intestinal cells is the first step of an infection. The pathogenic bacteria may colonise, cause cell damage and cross the epithelial membrane. Inhibition of pathogen adhesion in the gastro-intestinal tract is an important benefit provided by the pediococci of the invention. The pediococci strains and the pathogens compete with each other on the binding sites of epithelial cells and have been found able to prevent the binding of pathogens to the intestinal cells.

The pediococci strains, especially those isolated from human faeces, of the present invention that are able to produce pediocin are used as a probiotic. In the gastro-intestinal tract these strains will, after colonisation, produce pediocin and inhibit Gram-positive pathogens. In particular, pediocin produced from these strains has been found beneficial for inhibiting in the gastrointestinal tract the growth of Gram-positive pathogens of the enterococcus type.

Gram-positive species of the enterococci type that are preferentially inhibited in growth or otherwise by the pediococci according to the present invention are selected from vancomycin-resistant enterococci, Enterococcus faecalis, Enterococcus faecium, Listeria monocytogenes and mixtures thereof.

Separate to the growth inhibitory properties of its bacteriocin, pediococci strains of the invention, preferably the pediocin-producing pediococci, and more preferably those isolated from human faeces, have been found effective for inhibiting in the gastro-intestinal tract the adhesion and/or growth of Gram-negative strains.

Preferred Gram-negative species that are inhibited by the pediococci of the invention, in particular pediocin-producing pediococci and preferably P. acidilactici LMG P-21927, or by pediocin produced from the pediococci supernatant, are selected from the genera Klebsiella, Pseudomonas, Shigella and mixtures thereof, and more preferably are selected from Klebsiella pneumoniae, Pseudomonas aeruginosa, Shigella flexneri and mixtures thereof. That such pediocin-producing pediococci do provide adhesion inhibition of Gram-negative pathogens while its bacteriocin provides growth inhibition of Gram-positive pathogens is surprising. Indeed, it is not common that both the bacteriocin and the respective bacteria will have an effect so that inhibition of both Gram-positive and Gram-negative strains are inhibited.

Further, the pediocin-producing pediococci, preferably isolated from human faeces, as well as pediocin produced from these strains have been found effective for preventing associated disorders relating to the overgrowth of pathogens in the GI tract, in particular the gut, namely diarrhea but also associated secondary disorders selected from water disturbances, mineral balance disturbances, malnutrition, dysfunctioning of tissues, dysfunctioning of organs, dysfunctioning of organism, and mixtures thereof. The disturbances and malnutrition can in turn cause one or more of the following: nausea, vomiting, sickness but also endotoxemia, as well as sepsis (“blood poisoning”), whereas the dysfunctioning mentioned above can in turn cause one or more of the following: infections, ulcers, bad wound healing both in the gut as well as topically, and death.

Method of Isolation and Identification for the Pediocin-Producing Pediococci

a) Isolation

Faeces of healthy adult human volunteers are searched for probiotic strains. By “healthy”, it is meant an adult human having no illness, no affliction, not suffering from the GI tract diseases, not having used antibiotics for at least 6 weeks, not having consumed probiotic products for at least a week, not intolerant to milk proteins, and having regular bowel habits. A diary concerning dietary habits was recorded.

Fresh human faeces is analysed in an anaerobic chamber. The faeces is diluted tenfold in 90 ml of storage medium (20 g/l buffered peptone water, 1.0 ml/l Tween 80, 0.5 g/l L-cysteine-HCl and 1 Resazurin tablet per liter, pH 6.3 (adjusted with 2M HCl)) and then homogenised by using an Ultra-Turrax. Serial dilutions are made in reduced physiologic pepton water and the 10²-10⁷ dilutions are plated on LAMVAB (Hartemink et al. 1997, LAMVAB “A new selective medium for the isolation of lactobacilli from faeces, J. Microbiological methods 29, 77-84). The low pH (5.0) in this medium inhibits the growth of Gram-negative bacteria and the Gram-positive bacteria vancomycin-resistant Lactobacilli and pediococci are resistant to vancomycin, so LAMVAB is selective for these strains. This medium consist of 104.4 g/l De Man, Rogosa and Sharpe (MRS, Oxoid), 0.5 g/l L-cysteine-HCl, 0.05 g/l bromocresol green, 40 g/l agar, and 20 mg/l vancomycin. MRS, L-cysteine-HCl and bromocresol green are autoclaved separately from the agar for 15 minutes at 121° C. and cooled down to 50° C. Vancomycin is sterilised by filtration using a 0.2 μm filter. The three liquids are mixed together and plates are poured. The plates are incubated at 37° C. in anaerobic jars for three days. Gram-positive, catalase-negative rod and coc-shaped bacteria isolates are streaked for purity on MRS agar and incubated at 37° C.

It has also been found that a medium composed of:
i) a carbon and energy source that is not used by enterococci and the vast majority of lactobacilli but which is used by pediococci, and
ii) an antibiotic which does not inhibit Pediococcus but does inhibit the other genera such as an antibiotic derived from quinolones like Ciprofloxacin; and
iii) a small amount of pediocin to distinguish between a pediocin-producing Pediococcus and a non pediocin-producing Pediococcus
   provides a more selective medium for the determination of pediocin-producing Pediococci.

Preferably, the selective medium for pediocin-producing pediococci is a modified LAMVAB (Hartemink et al, 1997, as described hereinbefore) which contains 8 g/l of LAB-lemco powder (Oxoid) instead of meat extract given in LAMVAB, and a 2% xylose as carbon source instead of glucose. Xylose is separately autoclaved and added to the medium at 50° C. An antibiotic derived from quinolones such as Ciprofloxacin (10 mg/l) is added as an extra antibiotic and the supernatant of a steady state culture of P. acidilactici LMG P-21927 (10% w/v) is added to inhibit the non-pediocin-producing pediococci.

Accordingly, it is another aspect of the present invention to provide a method for isolating pediocin-producing Pediococci from a substrate, wherein the pediococci are isolated by using a medium comprising xylose, an antibiotic derived from quinolones, and pediocin. In a preferred embodiment the substrate is human faeces.

b) Selection for Bacteriocin Production

All isolated strains are then grown together with a positive control. Lactobacillus curvatus L530 in MRS for 24 hours at 37° C. Cells are removed by centrifugation (10 minutes, 4000 rpm, Sorval RT17), the pH of the supernatant is adjusted to 6.5 with NaOH and filter-sterilised. Indicator organisms were Bacillus cereus ATCC 11778, Enterococcus faecium ATCC 6569, Klebsiella pneumoniae LMD77-26, Listeria monocytogenes ATCC 7644, Pseudomonas aeruginosa DSM 1117 and Staphylococcus aureus ATCC 29213. These strains are grown overnight at 37° C. in BHI-broth. Sterile BHI-agar of 50° C. is inoculated with 1% of indicator organisms and plates are poured. After solidifying, wells of approximately 5 mm are made and 50 μl of sterile supernatant of the isolated strains is added. After overnight incubation at 37° C., the diameter of the clear zones around the wells is measured.

c) Species Identification

The API 50CHL (BioMerieux SA, France) is used for tentative identification of the strains by their fermentation profiles. Cells are grown overnight on MRS agar plates. Cells are removed from the agar plate with a sterile swab and resuspended in the suspension medium provided by the kit. API-strips are inoculated and analysed after 24 and 48 hours. Cells showing characteristics of a Pediococcus species are identified. Confirmation of cells being P. acidilactici, in particular P. acidilactici LMG P-21927 is confirmed with 16 sRNA sequencing.

d) 16sRNA

Sequencing of the 16sRNA gives a reliable identification of the strains. The extraction of the DNA of the strains is done according to the method described by Walter et al., 2000, “Detection and identification of gastrointestinal Lactobacillus species by using denaturing gradient gel electrophoresis and species-specific PCR primers”, Applied and Environmenal Microbiology, 66 (1), 297-303. The amplification and sequencing of the 16sRNA region is accomplished with primers mentioned in Table 1. The amplification program is 94° C. for 5 min; 30 cycles of 94° C. for 30 s, 54° C. for 30 s, 72° C. for 1 min 30 s; and finally 72° C. for 4 min.

TABLE 1
Sequence primers

| | |
|---|---|
| Sequence | |
| Primer | Sequence (5′→3′) |

| | |
|---|---|
| 8f | CAC GGA TCC AGA GTT TGA T(C/T)(A/C) |
| | TGG CTC AG |
| 338r | GCT GCC TCC CGT AGG AG |
| 338f | CTC CTA CGG GAG GCA GC |
| 515f | TGC CAG CAG CCG CGG TAA TAC GAT |
| 515r | ATC GTA TTA CCG CGG CTG CTG GCA |
| 968f | AAC GCG AAG AAC CTT AC |

Sequencing is carried out by the dideoxy method of Sanger et al., 1977, “DNA sequencing with chain-terminating inhibitors”, Proc. Natl. Acad. Sci. USA 74, 5463-5467, by using the ABI PRISM BigDye Terminator Cycle Sequencing Ready Reaction kit (Applied Biosystems Inc., Nieuwerkerk aan de IJssel, Netherlands) in combination with Applied Biosystems model 310 automated sequencing system. Analysis of nucleotide sequence data is carried out by using the Chromas/DNAsis program. The strain is identified with a BLAST search (NCBI), searching in the GenBank, EMBL, DDBJ and PDB databases.

Method of Identification of the Antimicrobial Agent

Protease Resistance:

To prove that the anti-microbial component is a protein, the activity of pediocin with addition of the enzyme protease K was tested with use of the well assay. Listeria monocytogenes ATCC 7644 was cultured 24 hours 37° C. and re-cultured overnight in BHI at 37° C. BHI was autoclaved with 12 g/l agar and cooled down to 50° C. 1% inoculum of indicator organism. Strain ATCC 7644 was added to the agar. Plates were poured and allowed to solidify. Wells of approximately 5 mm in diameter were made, and 50-100 μl of serial dilutions of the filter sterile neutral supernatant of P. acidilactici LMG P-21927 was dispensed in duplo into the wells. At one corner of the well 3 μl of (10 mg/ml) of protease K was added. After overnight incubation at 37° C., the diameter of the clear zones around the wells was measured. If the anti-microbial component is a protein or peptide, the component will be broken down and the clear zone will be absent on the place where the protease K was added. With this method it was demonstrated that the anti-microbial compound produced by strain LMG P-21927 was a protein or peptide.

Pediocin Plasmid Isolation and Sequence:

The plasmid with the pediocin-gene is isolated with use of the Birnboim method (Birnboim, H C and J. Doly, 1979, Nucleic Acid Res. 7:1513). The plasmid is multiplied with PCR, with use of the primers Pediocin 1 and Pediocin 2 (Table 2) and further purified with use of the GenElute PCR DNA purification kit (Sigma). A sequence PCR was done and the product is prepared for identification sequencing. After sequencing (use of the same primers, Table 2) the data is compared with data in the NCBI database. The plasmid of P. acidilactici LMG P-21927 has the gene that codes for pediocin PA-1/AcH.

TABLE 2
Primers pediocin

| | |
|---|---|
| Primer | Sequence (5′→3′) |

| | |
|---|---|
| Pediocin 1 | AAA ATA TCT AAC TAA TAC TTG |
| Pediocin 2 | TAA AAA GAT ATT TGA CCA AAA |

Form of Administration

Although the pediocin-producing pediococci according to the present invention can be used as such, it is preferred for use in the gastro-intestinal (GI) tract that the pediocin produced from Pediococcus is protected from its environment. Still for increased shelf-life storage, it may also be advantageous to protect the pediocin-producing pediococci strains. The type of protection is not limited and can include a coating, shell or encapsulation. The protection is designed for releasing its content, here the pediocin-producing pediococci, and/or pediocin produced from these strains, directly to its target. This enables the delivery of pediocin-producing pediococci, and/or pediocin produced from these strains in intact or almost intact form depending on the design of the protection to the GI tract, such as in the colon or, if a slow release system is used, in the whole GI tract.

A preferred mode of protection is by encapsulation. Method and material for encapsulation suitable for use herein are known in the art. Typical materials for encapsulation are selected from chitosan, maltodextrin, dextrins, lipids, polylactate, poly- or oligosaccharides, and mixtures thereof. Preferred encapsulating materials are selected from chitosan, maltodextrin, and mixtures thereof.

Accordingly, there is provided the use of pediocin-producing pediococci, and/or pediocin produced from these strains in a protected form, preferably in encapsulated form.

A typical mode of administration to the patient as well as to healthy persons of the pediocin-producing pediococci is enterally. A preferred enteral mode of administration is orally. Hence, when administered enterally, preferably orally, it is important that the strains or pediocin produced from these strains that are administered have a good survival rate so as to arrive in the small intestine with minimised destruction. By good survival, it is meant that at least 80% of the total ingested bacterial cells reaches the large intestine. The good survival is important as the strains have to survive the environment of the mouth, oesophagus, stomach, and finally small intestine but also when incorporated in products the strains have to survive the environment in the matrix of normal products that are meant for oral consumption. Of course, means of protection of the strains and/or bacteriocin as defined hereinbefore, such as encapsulation, can also be applied. It is still however important for the purpose of the invention that the strain fulfils the Survival Rate Test

Probiotic

The strain of pediococci according to the present invention and preferably Pediococcus acidilactici isolated from faeces such as LMG P-21927 has been found effective when used as a probiotic. Accordingly, a probiotic component is provided, wherein the probiotic comprises a pediococci according to the present invention and preferably comprises Pediococcus acidilactici isolated from faeces. Advantageously, the probiotic of the invention has a good survival rate in the stomach as well as in the intestine which is comparable to other known probiotics, such as L. rhamnosus ATCC 7469, L. rhamnosus ATCC 53103, L. plantarum DSM 9843, B. animalis from Chr. Hansen or better than known probiotics, such as L. acidophlilus La5 Chr. Hansen, L. reuteri LMG 9213.

Nevertheless, when better performance and/or shelf life of the invention probiotic is desired, the probiotic can be protected as described above, preferably by encapsulation.

Health-Promoting Action Composition

A health-promoting composition of the invention comprises a probiotic component, the probiotic component comprising a pediococcus according to the invention, and more preferably P. acidilactici isolated from human faeces LMG P-21927, and preferably further comprising one or more of the selected components: additional probiotic, prebiotics and immunoglobulins.

Alternatively, the probiotic component can be replaced by the pediocin produced from the probiotic strain or the pediocin can be used in combination with the probiotic in the health-promoting action composition of the invention. When used, the pediocin produced from the probiotic strain and present in the supernatant of the strain will be administered at a dosage of from 50 to 1000 millilitres pure supernatant per day, preferably from 200 to 500 ml. Additionally, the supernatant can be concentrated or the pediocin partly purified, subsequently leading to a lower volume dosage, dependent on the extent of concentration.

Preferably, the amount of probiotic component within the invention composition will be present in an amount of from 10⁶ to 5×10¹¹ cfu/day, more preferably from 10⁸ to 10¹⁰ cfu/day, optionally divided over e.g. 2, 3, 4, 5 or 6 dosage units per day.

Additional Probiotic

The use and the composition of the invention can comprise one or more additional probiotics. By use of an additional probiotic, the spectrum of activity can be further broadened or the activity of the pediocin-producing Pediococcus can even be increased. Preferred additional probiotics are selected from lactic acid bacteria, especially one or more strains from the genera Lactobacillus, Bifidobacterium, Propionibacterium. More preferred additional probiotics are L. rhamnosus, L. plantarum, B. animalis, B. lactis, L. fermentum, B. adolescentis, L. acidophilus, L. reuteri, B. longum, B. infantis, B. bifidum, B. breve, L. casei, L. johnsonii, L. gasseri, L. crispatus, L. helveticus, L. salivarius, L. lactis, L. brevis, L. paracasei, L. sakei and mixtures thereof.

When present, the amount of additional probiotic component within the invention composition will be present in an amount of from 10⁶ to 5×10¹¹ cfu/day, preferably from 10⁸ to 10¹⁰ cfu/day. The ratio (expressed in cfu) between the pediocin-prodcuing pediococci and the other pobiotics can be e.g. from 1:4 to 99:1.

Prebiotics

Prebiotics are substances that form a substrate for the probiotic compound. As a result, the likelihood that the microorganisms forming the probiotic component reach the intestines alive increases. Further, the combination of prebiotic with the probiotic enable increase of the beneficial action of the growth of beneficial bacteria such as probiotic components. Accordingly, prebiotics can advantageously be used in the invention composition. Suitable prebiotics for use herein are selected from trans-galactooligosaccharide, hydrolysed guarans (e.g. hydrolysed locust bean gum), inulin, hydrolysed inulin, fructooligosaccharides, xylooligosaccharides, xylopolysaccharides and mixture thereof. Preferred prebiotics are selected from inulin, hydrolysed inulin, fructooligosaccharides and mixtures thereof.

When present, the amount to be administered of prebiotic component will be of from 0.05 to 20 g/day, preferably from 0.2 to 10 g/day, most preferably from 0.5 to 5 g/day.

Immunoglobulins

Immunoglobulins are also suitable ingredients for use herein. Use of these in the invention will provide an additional or even synergistic effect on the reduction or inhibition of VRE. Preferably, the use of immunoglobulin Y from bird eggs is used. More preferably, the poultry has been hyperimmunised with pathogenic bacteria. Most preferably immunoglobulin Y derived from eggs of birds hyperimmunised against enterococci, Pseudomonas, Klebsiella and/or Shigella are used. Immunoglobulins can be added as in the egg fraction and/or be (partially) purified. Immunoglobulin Y interacts with the pathogenic bacteria in such a way that they are less able to colonise the GI tract of the host

When proteins are used that originate from eggs of hyperimmunised birds, the daily dose of IgY that can be administered is preferably from 0.2 to 1200 mg, more preferably within the range of 0.5 to 800 mg, most preferably from 10 to 600 mg. Stated otherwise, the dose of the ingredient is preferably of from 0.003 to 20 mg per dose per kg body weight, more preferably from 0.08 to 13 mg per dose per kg body weight, most preferably from 0.15 to 10 mg per dose per kg body weight.

Form of the Composition

The composition of the invention can be in any form that is suitable for its end use. This includes liquid, paste or solid form such as powder form. Any conventional pharmaceutically or nutritionally acceptable from can be used, such as tablets, coated tablets, capsules, granulates, elixirs, syrups, concentrated or diluted solutions or suspensions, sachets, suppositories, but also drinks, yoghurts, bonbons, bars, and other food products or food supplements.

Administration of the Invention Composition or Compound

The present invention is enterally administered. However, preferred modes of administration are by tube feeding or orally, more preferably oral administration.

### EXAMPLE 1

### Survival in Static Stomach and Small Intestine Model

The survival in the stomach and small intestine of P. acidilactici isolated from human faeces LMG P-21927 as well as known probiotic strains was evaluated. The survival of the pediococci in the stomach and small intestine is important when the strain is used as a probiotic in humans. Besides P. acidilactici LMG P-21927, known Pediococcus strains were tested: P. acidilactici ATCC 8081, P. acidilactici P-2 from a starter culture for meat fermentation from Christian Hansen, which P-2 produced pediocin, and P. acidilactici DSM 20284.

The bacteria were grown in MRS for 24 hours and subsequently re-inoculated for 18 hours in MRS. 1 ml of the grown culture was added to 9 ml of the stomach medium, consisting of 8.3 g/l bacteriological peptone, 3.1 g/l NaCl, 0.11 μl CaCl₂, 1.1 g/l KCl, 0.6 g/l KH₂PO₄, 22.2 mg/l pepsin and 22.2 mg/l lipase, pH 3.0. The bacteria were incubated for 3 hours at 37° C. in the stomach medium. Afterwards 1 ml of the incubated stomach medium with the bacterium was mixed with 9 ml of small intestine medium and incubated for another 3 hours at 37° C. The small intestine medium consists of 5.7 g/l bacteriological peptone, 1.25 g/l NaCl, 0.055 g/l CaCl₂, 0.15 g/l KCl, 0.68 g/l KH₂PO₄, 1.0 g/l NaHCO₃, 0.3 g/l Na₂HPO₄, 0.7 g/l glucose, 20.3 g/l pancreatin and 5.5 g/l bile, pH 6.5. Samples were taken at t=0, 3, and 6 hours and plated on MRS agar to determine the colony forming units.

It was found that P. acidilactici isolated from human faeces LMG P-21927 presented a better overall survival rate both in the stomach and small intestine according to the above Survival Rate Test than the P. acidilactici producing pediocin isolated from the other sources (Table 3). P. acidilactici DSM 20284 also shows a high survival rate, but disadvantageously is not able to produce pediocin.

TABLE 3
Survival of pediococci in stomach medium, small intestine medium and
according to the survival rate test (% of the colony forming units)*

| | | Survival | | |
|---|---|---|---|---|
| | | stomach | Survival small | Survival rate |
| Strain | Origin | medium | intestine medium | (overall) |
| P. acidilactici | Human | 92 | 111 | 102 |
| LMG P-21927 | faeces | | | |
| P. acidilactici | Fermented | 114 | 73 | 83 |
| ATCC 8081 | milk | | | |
| P. acidilactici | Meat starter | 65 | 76 | 49 |
| P-2 | culture | | | |
| P. acidilactici | Barley | 167 | 58 | 97 |
| DSM 20284 | | | | |

*The numbers for the survival in the small intestine medium (4^{th} column) are obtained after first incubation of the pediococci in stomach medium for 3 h, after which the survival was set to 100%.

### EXAMPLE 2

The P. acidilactici strains LMG P-21927, P-2, ATCC 8081 and DSM 20284 were grown in MRS for 24 hours and subsequently re-inoculated for 18 hours in MRS. The grown culture was 10 times diluted in PPS (Peptone physiological salt solution) (8.5 g/l sodium chloride, 1 g/l bacteriological peptone). 1 ml of this dilution was added to 9 ml of small intestine medium without pancreatin, with 5.5 or 1.1 g/l bile and incubated for 3 hours at 37° C. Samples were taken at t=0, and 3 hours and plated on MRS agar to determine the colony forming units. Results showed LMG P-21927 survived the small intestine with different bile salt concentrations better than the other pediococci (Table 4). This indicates that a pediococcus isolated from human faeces has better probiotic properties than a pediococcus isolated from food, meat and plants, since it has an improved ability to survive the conditions of the small intestine.

TABLE 4
Survival (in % of the colony forming units) of pediococci in small
intestine medium with 0.11%, and 0.55% Bile without prior incubation
in stomach medium

| Strain | Origin | 0.11 wt % | 0.55 wt % |
|---|---|---|---|
| LMGP-21927 | human faeces | 163 | 155 |
| ATCC 8081 | Fermented milk | 103 | 135 |
| P-2 (Christian Hansen) | Meat starter culture | 92 | 115 |
| DSM 20284 | Barley | 97 | 73 |

### EXAMPLE 3

### Growth on Prebiotics

P. acidilactici LMG P-21927 was grown in MRS for 24 hours and subsequently re-inoculated for 18 hours in MRS. Cultures were harvested by centrifugation (10 minutes 4000 rpm, Sorval RT 17) and the pellet was washed with and resuspended in PPS. This step was repeated. M17 medium (Oxoid) was prepared and different fibres were used as carbon source. The different M17 media were inoculated with 1% with the washed bacteria and incubated during 24 hours at 37° C. During incubation the optical density was measured. Results showed that P. acidilactici LMG P-21927 could grow on: trans-galactooligosaccharides (0.5% w/v), hydrolysed locust bean gum (0.5% w/v) and inulin, fructo-oligosaccharides and hydrolysed inulin (0.5% w/v).

### EXAMPLE 4

### Bacteriocin Activity

Growth and Production of Pediocin

The pediocin used for these studies was prepared as follows: P. acidilactici LMG P-21927 isolated from human faeces was grown for 24 hours 37° C. in MRS. 1% reinoculum in MRS was incubated for 24 hours at 37° C. The cells were removed by centrifugation (10 minutes, 4000 rpm, Sorval RT17). With NaOH the supernatant was set to pH 6.5 and after that filter-sterilised and frozen (−20° C.) until use. Supernatant of P. acidilactici DSM 20284 was used as a control, because this strain did not produce pediocin.

Detection of the Antimicrobial Activity

The bacteria tested for sensitivity for P. acidilactici LMG P-21927 were Gram-positive pathogens Listeria monocytogenes ATCC 7644, Enterococcus faecium (ATCC 6569-non VRE), Enterococcus faecium (LMG 21895, LMG 21896, LMG 21897, ATCC 700221, LMG 21898, LMG 21899, LMG 21900 which are all VRE clinical isolates of E. faecium), E. faecium ATCC 700221 (VRE), E. faecalis (ATCC 4200, LW603, ATCC 376, ATCC 14428, ATCC 29212, all non VRE) and commensal bacteria and probiotic strains Lactobacillus rhamnosis ATCC 7469, L. acidophilus LW 74-2, L. casei DSM20011, L. plantarum DSM20174, Lactococcus lactis LW53, Bifidobacterium longum BB536, B. breve ATCC 15700, B. animalis Bb12 from Christian Hansen, B. adolescentis ATCC 15705 and B. bifidum DSM20456.

The pathogens are grown in Brain Heart Infusion (BHI, Oxoid) and the lactobacilli, lactococci and bifidobacteria in MRS. The bifidobacteria were grown in an anaerobic chamber in MRS with 0.5 μl cysteine-HCl and 1 resazurin tablet per liter medium. The antimicrobial activity of P. acidilactici LMG P-21927 was detected using a well diffusion assay and by measuring the optical density (OD) during growth. The bifidobacteriae were only tested with the well diffusion assay, because of difficulties in measuring the OD under anaerobic conditions.

In the well diffusion assay the bacteria were grown in their standard medium for 24 hours at 37° C. and re-cultured overnight at 37° C. Suitable media were autoclaved with 12 g/l agar and cooled down to 50° C. 1% inoculum of the to be tested bacteria was added to the agar. Plates were poured and allowed to solidify. Wells of approximately 5 mm in diameter were made, and 50-100 μl of serial dilutions of the filter sterile neutral supernatant of P. acidilactici LMG P-21927 was dispensed in duplicate into the wells. After overnight incubation at 37° C., the diameter of the clear zones around the wells was measured.

In the other method the OD at 600 nm was measured in time for bacteria incubated with supernatant of P. acidilactici LMG P-21927. Bacteria were grown in their normal medium for 24 hours at 37° C. and re-inoculated 1% in the same medium for 18 hours at 37° C. 125 μl of supernatant of P. acidilactici LMG P-21927 was mixed in a 100-wells-plate with an equal amount of double concentrated media of the bacteria to be tested. The bacteria were added so that the end concentration in the plates was 10⁵ cfu/ml. OD₆₀₀ₙₘ was measured during 24 hours at 37° C. with the use of a Bioscreen apparatus. Bacterial growth with supernatant of P. acidilactici DSM 20284 was used as a control. Experiments were done in duplicate.

Results

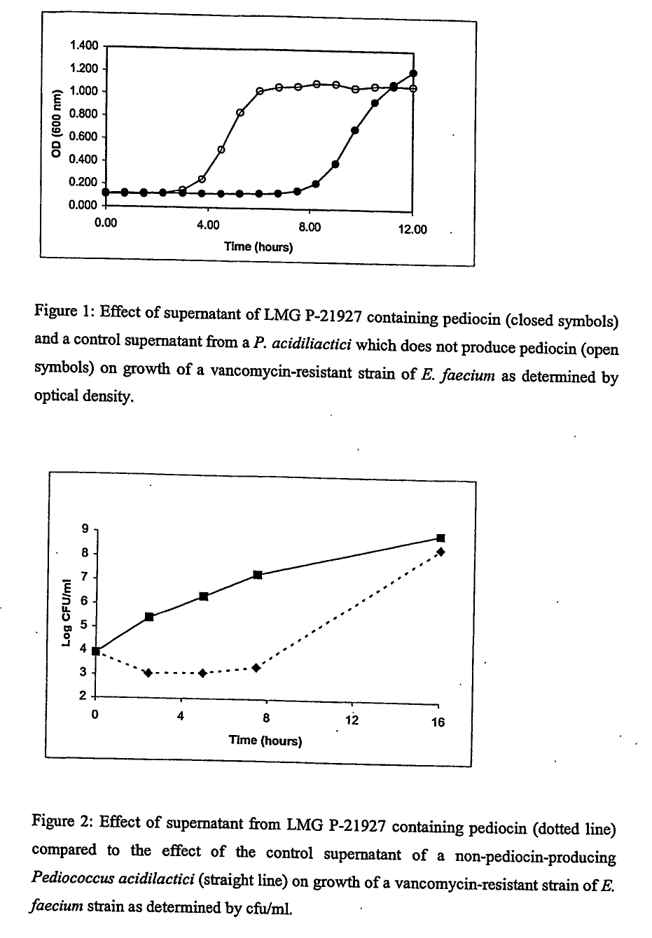

Listeria monocytogenes ATCC 7644 and E. faecium strains both VRE and non-VRE and E. faecalis strains were sensitive for supernatant of P. acidilactici LMG P-21927. L. monocytogenes ATCC 7644 was inhibited by P. acidilactici LMG P-21927 by lowering the growth rate and E. faecium had a much longer lag-phase than without the supernatant (FIG. 1). Commensal and probiotic strains such as lactobacilli and bifidobacteria were not sensitive for P. acidilactici LMG P-21927 and its pediocin.

To measure if the effect of P. acidilactici LMG P-21927 on the bacteria was bactericidal or bacteriostatic time-kill curve studies were done. Supernatant of P. acidilactici LMG P-21927 was mixed 1:1 with 2 times concentrated BHI. 10⁵ cfu/ml of L. monocytogenes ATCC 7644 or VRE E. faecium (LMG 21895, LMG 21896, LMG 21897), E. faecium ATCC 700221 VRE was added to medium and supernatant. Samples were taken during appropriate intervals for a period of 24 hours period to determine the viable cell count

The effect of P. acidilactici on bacteria is bactericidal. The amount of E. faecium is decreased 1 Log unit after adding supernatant. (FIG. 2).

### EXAMPLE 5

### Adhesion of P. acidilactici to Caco-2 Cells Compared with Known Probiotic Strains

One of the properties of probiotics is that they can adhere to intestinal cells and compete with pathogens for the binding sites of the epithelial cells.

The adherence of well known, good adhering probiotic strains Lactobacillus rhamnosus ATTC 53103 (L GG), and Bifidobacterium animalis Bb-12 (Christian Hansen), and of P. acidilactici LMG P-21927 was tested.

Overnight cultures of the strains were harvested by centrifugation (10 minutes, 4000 rpm, Sorval RT17) and resuspended in PBS. The amount of cells was counted under a microscope with use of a Bürker Türk counting chamber. Bacteria were centrifuged again and the pellet was resuspended in Caco-2 1% FCS-medium Pen/Strep free. The Caco-2 cells were 2 weeks post-confluence and grown in a 24 wells-plate (2.5 10⁵ Caco-2 cells per well). Per well 2.5 10⁸ CFU of the bacteria were added and incubated for 1 hour at 37° C. in an incubator with 5% CO₂. After incubation the media was removed from the Caco-2 cells and the cells were washed 3 times with PBS (37° C.). Cells were lysed with sterile Mili Q water, serial dilutions of the lysed cells were made and plated on MRS agar. Results showed that strain LMG P-21927 adhered better than other probiotic strains (Table 5).

TABLE 5
Adhesion of protiotics to Caco-2 cells.

| | Adhesion |
|---|---|
| | (% of total bacterial cells added) |
| P. acidilactici LMG P-21927 | 0.9 |
| L. rhamnosus ATTC 53103 | 0.14 |
| B. Animalism Bb-12 (Chr. Hansen) | 0.66 |

### EXAMPLE 6

### Inhibition of Adhesion and/or Invasion of Pathogenic Bacteria by P. acidilactici LMG P-21927

Interaction between pathogenic bacteria and the host cells initiates infectious diseases. Attachment of these pathogens to intestinal cells is the first step of an infection. The pathogenic bacteria may colonise, cause cell damage and when invasive cross the epithelial membrane. Probiotic strains and pathogens compete with each other on the binding sites of epithelial cells. Probiotic strains can prevent the pathogens in binding to the intestinal cells.

Using cultured Caco-2 cells as a human intestinal cell model, the adhesion of pathogens and probiotic strains to cells (Coconnier, et el., 1993) was tested. Pathogens tested were Klebsiella pneumoniae LMG 21902, Pseudomonas aeruginosa LMG 21901 and Shigella flexneri LMG 21935, all deposited at the BCCM™/LMG.

The probiotics used were P. acidilactici LMG P-21927 and the commercial used strain Lactobacillus rhamnosus ATTC 53103 and Bifidobacterium animalis Bb-12.

Overnight cultures of pathogens and probiotics were harvested by centrifugation (10 minutes, 4000 rpm, Sorval RT17) and resuspended in PBS. The amount of cells was counted under a microscope with use of Bürker Türk counting chamber. Bacteria were centrifuged again and the pellet was resuspended in Caco-2 1% FCS-medium Pen/Strep free. The Caco-2 cells were 2 weeks post-confluence and grown in a 24 wells-plate (2.5 10⁵ Caco-2 cells per well). Per well 5 10⁷ CFU of the pathogens and 2.5 10⁸ CFU of the probiotic bacteria were added and incubated for 1 hour at 37° C. in an incubator with 5% CO₂. After incubation the media was sucked off from the Caco-2 cells and the cells were washed 3 times with PBS (37° C.). Cells were lysed with sterile Mili Q water, serial dilutions of the lysed cells were made and plated on Nutrient agar (for pathogens) and on MRS agar (for probiotic strains).

P. acidilactici LMG P-21927 was found to reduce the adherence of the pathogens to intestinal cells. LMG P-21927 was found as good in prevention of adhesion as well known probiotics (Table 6).

TABLE 6
Inhibition of adhesion of pathogens to Caco-2 cells by probiotics (%).

| | K. pneumonia | P. aerruginosa | S. flexneri LMG |
|---|---|---|---|
| | LMG 21902* | LMG 21901* | 21935* |
| P. acidilactici | 40 | 35 | 25 |
| LMG P-21927 | | | |
| L. rhamnosus LGG | 44 | 38 | Nd |
| ATTC 53103 | | | |
| B. Animalism Bb-12 | Nd | 42 | Nd |
| (Chr. Hansen) | | | |

*The prevention (%) of the adhesion of the Caco-2 cells by probiotic bacteria: Difference in adherence of the pathogens with and without probiotic strains. Nd = not determined.

### EXAMPLE 7

A product in the form of a sachet containing 1 g of maltodextrin and 5×10⁹ cfu LMG P-21927. The sachet product is to be taken twice a day.

The contents of the sachet are intended for addition to a beverage or dessert and mixed upon consumption. The product, to which it is added is cold or lukewarm, but does not have a temperature exceeding 45° C.

### EXAMPLE 8

Milk powder or infant milk formula (powder) containing 1×10⁷ cfu LMG P-21927 per gram. The powder further contains a probiotic belonging to the genus Bifidobacterium in a concentration of 1×10⁷ cfu/g.

### EXAMPLE 9

Capsule, with an acid resistant coating, containing 2×10⁹ cfu LMG P-21927, 0.4 g of hydrolysed inulin and 0.1 g of protein derived from eggs of chickens, which were hyperimmunised against a cocktail of vancomycin-resistant Enterococci strains.

### EXAMPLE 10

Milk powder, obtained after fermentation of skim milk, supplemented with 0.5 wt % glucose, and LMG P-21927 at a pH kept constant at 6.0. After 48 h of fermentation at 37° C., the fermented milk is dried by processes known in the art.

### EXAMPLE 11

Probiotic bar of 23 gram containing 4.0 g oat flakes, 3.0 g wheat flakes, 3.0 g puffed rice, 1.0 g crushed hazelnuts, 0.25 g raisins, 1.5 g maltodextrin, and 2×10⁹ cfu LMG P-21927.

### EXAMPLE 12

Isotonic drink with a sealed cap that contains 1.0 gram maltodextrin and 2×10⁹ cfu LMG P-21927.

1-16. (canceled)

17. A method for inhibiting the growth of pathogenic strains in the gastrointestinal tract in a human in need thereof, comprising administering pediocin-producing pediococci to said human, wherein the pediococci are characterized by a survival rate as per the Survival Rate Test defined herein of at least 80%.

18. The method according to claim 17, wherein the survival rate is at least 90%.

19. The method according to claim 17, wherein the pediococci are isolated from human faeces.

20. The method according to claim 17, wherein said administration prevents and/or treats infections by pathogenic bacteria in the gastrointestinal tract, diarrhoea, and/or secondary disorders associated herewith.

21. The method according to claim 17, wherein the secondary disorders are selected from the group consisting of water disturbances, mineral balance disturbances, malnutrition, dysfunctioning of tissues, dysfunctioning of organs, dysfunctioning of organism, and combinations thereof.

22. The method according to claim 17, wherein the pediococci comprise Pediococcus acidilactici strain LMG P-21927.

23. The method according to claim 17, wherein the pathogenic bacteria are Gram-negative bacteria.

24. The method according to claim 17, wherein the pathogenic bacteria are Gram-positive bacteria.

25. The method according to claim 17, comprising administering pediocin-producing pediococci in combination with one or more further probiotics selected from the group consisting of Lactobacillus rhamnosus, L. plantarum, L. fermentum, L. acidophilus, L. reuteri, L. casei, L. johnsonii, L. gasseri, L. crispatus, L. helveticus, L. salivarius, L. lactis, L. brevis, L. paracasei, L. sakei, Bifidobacterium animalis, B. lactis, B. adolescentis, B. longum, B. infantis, B. bifidum and B. breve.

26. A method for inhibiting the growth of pathogenic bacteria in the gastrointestinal tract in a human in need thereof, comprising administering pediocin-producing pediococci to said human, wherein the pediococci are isolated from human faeces.

27. The method according to claim 26, wherein said administration prevents and/or treats infections by pathogenic bacteria in the gastrointestinal tract, diarrhoea, and/or secondary disorders associated herewith.

28. The method according to claim 26, wherein the secondary disorders are selected from the group consisting of water disturbances, mineral balance disturbances, malnutrition, dysfunctioning of tissues, dysfunctioning of organs, dysfunctioning of organism, and combinations thereof.

29. The method according to claim 26, wherein the pediococci comprise Pediococcus acidilactici strain LMG P-21927.

30. The method according to claim 26, wherein the pathogenic bacteria are Gram-negative bacteria.

31. The method according to claim 26, wherein the pathogenic bacteria are Gram-positive bacteria.

32. The method according to claim 26, comprising administering pediocin-producing pediococci in combination with one or more further probiotics selected from the group consisting of Lactobacillus rhamnosus, L. plantarum, L. fermentum, L. acidophilus, L. reuteri, L. casei, L. johnsonii, L. gasseri, L. crispatus, L. helveticus, L. salivarius, L. lactis, L. brevis, L. paracasei, L. sakei, Bifidobacterium animalis, B. lactis, B. adolescentis, B. longum, B. infantis, B. bifidum and B. breve.

33. An isolated pediocin-producing pediococcus characterised by a survival rate as per the Survival Rate Test defined herein of at least 90%.

34. The pediococcus according to claim 33, wherein the pediococcus is Pediococcus acidilactici as deposited at BCCM™/LMG under No. LMG P-21927.

35. A health-promoting composition comprising a pediococcus according to claim 33, as a probiotic component.

36. The health-promoting composition according to claim 35, further comprising a component selected from the group consisting of pediocin, additional probiotics, prebiotics, immunoglobulins, and mixtures thereof.

37. A method for isolating pediocin-producing pediococci from a substrate, wherein the pediocin-producing pediococci is isolated by using a medium comprising xylose, an antibiotic derived from quinolones and pediocin.

38. The method according to claim 37, wherein the substrate is human faeces.


SEQUENCE LISTING (8 sequences)

SEQ ID NO: 1 - DNA, 29 bp, Pediococcus acidilactici
  misc_feature: sequence primer P. acidilactici
cacggatccagagtttgatymtggctcag

SEQ ID NO: 2 - DNA, 17 bp, Pediococcus acidilactici
  misc_feature: sequence primer P. acidilactici
gctgcctcccgtaggag

SEQ ID NO: 3 - DNA, 17 bp, Pediococcus acidilactici
  misc_feature: sequence primer P. acidilactici
ctcctacgggaggcagc

SEQ ID NO: 4 - DNA, 24 bp, Pediococcus acidilactici
  misc_feature: sequence primer P. acidilactici
tgccagcagccgcggtaatacgat

SEQ ID NO: 5 - DNA, 24 bp, Pediococcus acidilactici
  misc_feature: sequence primer P. acidilactici
atcgtattaccgcggctgctggca

SEQ ID NO: 6 - DNA, 17 bp, Pediococcus acidilactici
  misc_feature: sequence primer P. acidilactici
aacgcgaagaaccttac

SEQ ID NO: 7 - DNA, 21 bp, Pediococcus acidilactici
  misc_feature: sequence primer P. acidilactici
aaaatatctaactaatacttg

SEQ ID NO: 8 - DNA, 21 bp, Pediococcus acidilactici
  misc_feature: sequence primer P. acidilactici
taaaaagatatttgaccaaaa